(19) United States
(12) Patent Application Publication
(10) Pub. No.: US 2015/0164968 A1
(43) Pub. Date: Jun. 18, 2015

(54) Method of Producing Banana-Derived Composition and Biologically Active Substance Containing Same

(71) Applicant: Masaru Otani, Tokyo (JP)

(72) Inventor: Masaru Otani, Tokyo (JP)

(21) Appl. No.: 14/540,263

(22) Filed: Nov. 13, 2014

(30) Foreign Application Priority Data
Nov. 14, 2013 (JP) 2013-236183
Oct. 7, 2014 (JP) 2014-206694

(51) Int. Cl. A61K 36/88; C12P 21/06; A61K 31/4045; A61K 31/198; A61K 31/405

(52) U.S. Cl. CPC A61K 36/88; A61K 31/198; A61K 31/405; A61K 31/4045; C12P 21/06

(57) ABSTRACT

Provided is a method of producing a banana-derived composition by which a composition exhibiting excellent biological activity is obtained from a flesh part of a banana. The method includes the steps of (1) mincing a flesh part of a banana to obtain flesh fractions; (2) adding a proteolytic enzyme to the flesh fractions and decomposing proteins contained in the flesh fractions through an enzyme reaction, to obtain an enzymatic reactant; and (3) deactivating the proteolytic enzyme contained in the enzymatic reactant to obtain a composition A.

### CROSS-REFERENCE TO RELATED APPLICATION

This application claims priority to and the benefit of Japanese Patent Application No. 2013-236183 filed on Nov. 14, 2013 and Japanese Patent Application No. 2014-206694 filed on Oct. 7, 2014, the entire contents of which are incorporated herein by reference.

### TECHNICAL FIELD

The present invention relates to a method of producing a banana-derived composition and a biologically active substance containing a banana-derived composition.

### BACKGROUND

Bananas, which belong to the Musaceae family, are widely grown around the world. In recent years, parts such as a flower and a root of a banana have been found to exhibit a variety of biological activity, which is the center of public attention. Examples of the biological activity include, for example, an action of inhibiting a blood sugar level rise. The recent number of adult patients with diabetes has reached several billions worldwide. As a method for inhibiting the blood sugar level rise and curing diabetes, for example, insulin administration, pancreatic islet transplantation, and exercises are considered to be effective. On the other hand, extracts of banana flowers and roots have been also reported to contribute to improve a hyperglycemic state. (Refer to, for example, Non-Patent Literatures 1 and 2.) For example, Non-Patent Literature 1 reports that a banana root extract has an antidiabetic effect. Non-Patent Literature 2 also reports that a banana flower extract has an antihyperglycemic effect.
Non-Patent Literature 1: Salau B. A et al., Asian J. Exp. Biol. Sci., vol. 1 (1): pp. 30-35, 2010.
Non-Patent Literature 2: L. Pari and J. Umamaheswari, Phytother. Res, vol. 14: pp. 136-138, 2000.

### SUMMARY OF INVENTION

However, banana roots and flowers are not easily available, and there has been a demand for a supply at a more reasonable price and in a more reliable manner. Furthermore, a mechanism of banana roots and flowers providing the biological activity, such as the action of inhibiting the blood sugar level rise, is not known.

The present invention has been conceived in view of the above circumstances, and an objective of the present invention is to provide a method of producing a banana-derived composition by which a composition exhibiting excellent biological activity is obtained from a flesh part of a banana. Another objective of the present invention is to provide a biologically active substance that exhibits excellent biological activity, the biologically active substance containing a banana-derived composition obtained from a flesh part of a banana.

The present inventor has focused on a flesh part of a banana that has an established distribution channel in the market and that is available at a reasonable price in a considerable amount and conducted an earnest studies to achieve the above objective. As a result, the present inventor has found that a composition obtained by enzymolysis of proteins contained in the flesh part of the banana exhibits excellent biological activity, thus achieving the present invention.

That is to say, the present invention is to solve the aforementioned problems advantageously, and one aspect of the present invention resides in a method of producing a banana-derived composition, the method including the steps of: (1) mincing a flesh part of a banana to obtain flesh fractions; (2) adding a proteolytic enzyme to the flesh fractions and decomposing proteins contained in the flesh fractions through an enzyme reaction to obtain an enzymatic reactant; and (3) deactivating the proteolytic enzyme contained in the enzymatic reactant to obtain a composition A. By producing a banana-derived composition according to the method including the above steps, the produced banana-derived composition has the excellent actions of inhibiting the blood sugar level rise and promoting collagen production.

In the above method of producing a banana-derived composition according to the present invention, it is preferable that the flesh part of the banana includes a flesh part of an unripe banana. By using the flesh part of an unripe or a green banana as the flesh part of the banana, the produced banana-derived composition has excellent biological activity such as the actions of inhibiting the blood sugar level rise and promoting collagen production. Moreover, by doing so, particularly when alcohol having a carbon number of 1 to 4 is used for the deactivation of the proteolytic enzyme, production efficiency is significantly improved.

In the above method of producing a banana-derived composition according to the present invention, it is also preferable that the step (3) deactivates the proteolytic enzyme is deactivated by heating. The deactivation of the proteolytic enzyme by heating requires only a simple operation and a simple facility, thereby improving production efficiency.

In the above method of producing a banana-derived composition according to the present invention, it is also preferable that the step (3) deactivates the proteolytic enzyme with alcohol having a carbon number of 1 to 4, and includes the steps of: (i) adding the alcohol having the carbon number of 1 to 4 to the enzymatic reactant, and subsequently subjecting a resulting alcohol-added substance to solid-liquid separation to extract a liquid phase; and (ii) removing the alcohol having the carbon number of 1 to 4 from the liquid phase. By deactivating the proteolytic enzyme with the alcohol having the carbon number of 1 to 4, an active ingredient is extracted by the alcohol correspondingly, and a yield of the obtained active ingredient is improved.

In the above method of producing a banana-derived composition according to the present invention, it is also preferable that the alcohol having the carbon number of 1 to 4 includes at least one of methanol and ethanol. Methanol and ethanol, which have a lower boiling point than other types of alcohol, are easy to remove by evaporation. Use of methanol and ethanol with the above property helps reduce a thermal hysteresis of the banana-derived composition to be produced, resulting in improved biological activity of the composition. Furthermore, methanol and ethanol have a high efficiency of extraction of an amino acid and a tryptophan derivative that are contained in the flesh part of the banana.

In the above method of producing a banana-derived composition according to the present invention, it is also preferable that the step (ii) includes the step of removing the alcohol having the carbon number of 1 to 4 from the liquid phase by freeze drying. By adopting freeze drying as a method for removing the alcohol, a thermal hysteresis of the banana-derived composition to be produced is reduced, resulting in improved biological activity of the composition.

The above method of producing a banana-derived composition according to the present invention preferably further includes the step of: (4) desugaring and desalting the composition A to obtain a composition B. With the above step, the produced banana-derived composition has the excellent actions of inhibiting the blood sugar level rise and promoting collagen production, and moreover, the produced banana-derived composition has an excellent action of antioxidation.

A banana-derived composition produced by the method of producing a banana-derived composition according to the present invention exhibits biological activity.

The present invention is to solve the aforementioned problems advantageously, and another aspect of the present invention resides in a biologically active substance, including: as an active ingredient, a banana-derived composition obtained from a flesh part of a banana. The biologically active substance according to the present invention has excellent biological activity.

In the above biologically active substance according to the present invention, it is preferable that the banana-derived composition is obtained from a flesh part of an unripe banana. When the banana-derived composition is obtained from the flesh part of an unripe banana, the biological activity of the biologically active substance according to the present invention is improved.

In the above biologically active substance according to the present invention, it is also preferable that the banana-derived composition is obtained by adding a proteolytic enzyme to the flesh part of the banana to decompose proteins contained in the flesh part of the banana through an enzyme reaction, and subsequently deactivating the proteolytic enzyme by heating or treating with alcohol. When the banana-derived composition is obtained through the enzyme reaction and through the deactivation by heating or with alcohol, the biological activity of the biologically active substance according to the present invention is improved.

In the above biologically active substance according to the present invention, it is also preferable that the banana-derived composition contains an amino acid and a tryptophan derivative, and a total amount of the amino acid and the tryptophan derivative is 7% by mass or more. When the total amount of the amino acid and the tryptophan derivative contained in the banana-derived composition is 7% by mass or more, the biological activity of the biologically active substance according to the present invention is improved.

In the above biologically active substance according to the present invention, it is also preferable that the amino acid includes phenylalanine and tryptophan, and the tryptophan derivative includes serotonin. When the biologically active substance includes phenylalanine, tryptophan, and serotonin, the biological activity of the biologically active substance according to the present invention is improved.

The above biologically active substance according to the present invention exhibits at least one of the action of inhibiting a blood sugar level rise and the action of promoting collagen production.

According to the present invention, a method of producing a banana-derived composition by which a composition exhibiting excellent biological activity is obtained from a flesh part of a banana is provided. Furthermore, according to the present invention, a biologically active substance containing a banana-derived composition obtained from a flesh part of a banana that exhibits excellent biological activity is provided.

### BRIEF DESCRIPTION OF DRAWINGS

The present invention will be further described below with reference to the accompanying drawings, wherein:

FIG. 1 illustrates a result of measurement of an action of inhibiting the blood sugar level rise (in rats);

FIG. 2 illustrates a result of measurement of an action of inhibiting the blood sugar level rise (in a human);

FIG. 3 illustrates a result of measurement of an action of inhibiting a blood sugar level rise (in a human); and

FIG. 4 illustrates a result of measurement of an action of promoting collagen production.

### DESCRIPTION OF EMBODIMENTS

Embodiments of the present invention will be described in detail below. The present invention is directed to a method of producing a banana-derived composition by which a composition having excellent biological activity is produced by using a flesh part of a banana as a material. The present invention is also directed to a biologically active substance including, as an active ingredient, a banana-derived composition obtained from a flesh part of a banana. The banana-derived composition included in the biologically active substance according to the present invention may be produced by the method of producing a banana-derived composition according to the present invention.

### <Method of Producing Banana-Derived Composition>

The method of producing a banana-derived composition according to the present invention includes the steps of: (1) mincing a flesh part of a banana to obtain flesh fractions; (2) adding a proteolytic enzyme to the flesh fractions and decomposing proteins contained in the flesh fractions through an enzyme reaction to obtain an enzymatic reactant; and (3) deactivating the proteolytic enzyme contained in the enzymatic reactant to obtain a composition A. The above method of producing a banana-derived composition according to the present invention may further include, in addition to the steps (1) to (3), the step of: (4) desugaring and desalting the composition A to obtain a composition B.

The banana-derived composition obtained by the above method exhibits excellent biological activity. Although a mechanism of how the banana-derived composition obtained by the method of producing a banana-derived composition according to the present invention has the excellent biological activity is yet unknown, it is inferred that an amino acid (preferably, phenylalanine and tryptophan) and a tryptophan derivative (preferably, serotonin) that are obtained mainly through an enzyme reaction contribute to the biological activity (the actions of inhibiting the blood sugar level rise, promoting collagen production, and antioxidation). The method according to the present invention is capable of efficiently extracting the amino acid and the tryptophan derivative and producing the banana-derived composition containing the amino acid and the tryptophan at a high concentration.

Examples of the amino acid that may be contained in the produced banana-derived composition include, but are not limited to, arginine, lysine, histidine, phenylalanine, tyrosine, leucine, isoleucine, methionine, valine, alanine, glycine, proline, glutamic acid, serine, threonine, asparagine acid, and tryptophan.

The tryptophan derivative refers to a compound (or a substance) other than the amino acid that may be derived from tryptophan, preferably a compound (or a substance) other than the amino acid that may be derived from tryptophan in a biological reaction in a human body. Examples of the tryptophan derivative include, but are not limited to, serotonin and melatonin.

In the description herein, the “step (1) of mincing a flesh part of a banana to obtain flesh fractions” may be simply referred to as the “flesh mincing step (1)”, the “step (2) of adding a proteolytic enzyme to the flesh fractions for decomposing proteins contained in the flesh fractions through an enzyme reaction to obtain an enzymatic reactant” may be simply referred to as the “enzyme reaction step (2)”, and the “step (3) of deactivating the proteolytic enzyme contained in the enzymatic reactant to obtain a composition A” may be simply referred to as the “enzyme deactivation step (3)”, and the “step (4) of desugaring and desalting the composition A to obtain a composition B” may be simply referred to as the “desugaring and desalting step (4)”, as appropriate.

### [Flesh Mincing Step (1)]

### (Mincing)

In the method of producing a banana-derived composition according to the present invention, firstly, a flesh part of a banana is fractioned to obtain flesh fractions. Mincing the flesh part of the banana into the flesh fractions facilitates efficient reaction of the proteolytic enzyme with proteins in the enzyme reaction step (2) which is later described. It is noted that the term “mincing” as used herein refers to fragmentation of a flesh part, being in an unbroken state after a peel thereof is pulled off, into pieces each having a mass that is smaller than a mass in the above state, or to turning the flesh part into a paste by crushing, chopping, or the like. Shapes of the flesh fractions resulting from the mincing are not particularly limited and may be in the form of granules or a paste. However, from the viewpoint of satisfactorily ensuring efficiency of the enzyme reaction, a pasty form is preferable.

When the resulting flesh fractions are in the form of granules, an average mass of the fractions is preferably 20 gram or less, more preferably 15 gram or less, and even more preferably 10 gram or less. The average fraction mass of 20 gram or less satisfactorily ensures the efficiency of enzyme reaction. Although the average mass of the fractions is not particularly limited, the average mass of the fractions is typically 0.1 gram or more. When the number of the fractions resulting from the mincing is greater than 10, the “average mass of the fractions” may be obtained as an average value of masses of ten fractions that are arbitrarily selected. When the number of the fractions resulting from the mincing is less than or equal to 10, the “average mass of the fractions” may be obtained as an average value of masses of all the fractions.

A method for mincing the flesh part of a banana is not particularly limited, and a method using a cutter mill crusher, a hammer mill crusher, or the like is considered.

### (Flesh Part of Banana)

In the present invention, the flesh part of the banana refers to a part (which corresponds to a so-called edible part when the banana is an edible banana) obtained by pulling off the peel of the banana fruit. The variety of the banana to be used is not particularly limited and may be the Giant Cavendish variety, the Rakatan variety, or the like. A single variety or two or more varieties may be used. Among the varieties, the Giant Cavendish is most preferable. The flesh part of the banana may be obtained by pulling off the peel with use of a known method.

In the present invention, the flesh part of the banana is preferably the flesh part of an unripe banana. It is noted that the “unripe banana” as used herein refers to the one whose fruit juice has a Brix degree of less than 24° Bx as measured by means of a sugar refractometer. The fruit juice is obtained by, for example, crushing or chopping the flesh part into a paste and feeding the paste to a filter press. By using the flesh part of such an unripe banana that has a relatively small content of sugar as the flesh part of the banana, the amount of sugar contained in the obtained banana-derived composition (a primary refined product) is reduced. As a result, the amount (ratio) of the amino acid and the tryptophan derivative (preferably, phenylalanine, tryptophan, and serotonin) contained in the obtained banana-derived composition is increased. That is to say, when the flesh part of an unripe banana is used, compared with a case using the flesh part of a ripe banana, a total amount of the amino acid and the tryptophan derivative contained in the banana-derived composition is increased. As a result, the biological activity (the actions of inhibiting the blood sugar level rise and promoting collagen production) is improved. Furthermore, the flesh part of an unripe banana has a small viscosity. Accordingly, by using the flesh part of an unripe banana as the flesh part of the banana, efficiency of filtration or the like in a manufacturing process is improved (for example, extraction of a liquid phase in the solid-liquid separation step (i) which is later described is facilitated). As a result, production efficiency is significantly improved.

From the viewpoints of thus improving production efficiency, facilitating the desugaring, and improving the biological activity, the Brix degree of the flesh part of the banana is preferably 10° Bx or less, more preferably 8° Bx or less, and even more preferably 5° Bx or less, and most preferably 2° Bx or less. Although the lower limit of the Brix degree in the flesh part of the banana is not particularly limited, the Brix degree is typically 0.1° Bx or more.

### [Enzyme Reaction Step (2)]

Subsequently, a proteolytic enzyme is added to the obtained flesh fractions for decomposing proteins contained in the flesh fractions through the enzyme reaction to obtain an enzymatic reactant. The enzyme reaction significantly increases the amount of the amino acid and the tryptophan derivative (preferably, phenylalanine, tryptophan, and serotonin) contained in the banana-derived composition to be produced, and therefore, the biological activity is ensured. The “enzyme reaction” herein includes fermentation using microorganisms.

### (Proteolytic Enzyme)

The proteolytic enzyme used in the enzyme reaction step (2) is not particularly limited as long as the proteolytic enzyme is capable of decomposing proteins contained in the flesh part of the banana, and either an endo-type or an exo-type enzyme may be used. Examples of the proteolytic enzyme agent including the end-type proteolytic enzyme include Protin SD-AY10 and Protin SD-NY10 (both of which are manufactured by Amano Enzyme Inc.). Examples of the proteolytic enzyme agent including the exo-type proteolytic enzyme include ProteAX™, Protease M “Amano” SD, Protease P “Amano” SD (all of which are manufactured by Amano Enzyme Inc.).

In terms of the capability of efficiently decomposing proteins contained in the flesh fractions and generating the amino acid, the proteolytic enzyme to be used is preferably the exo-type proteolytic enzyme, and is more preferably Protease M “Amano” SD.

### (Enzyme Reaction)

In the enzyme reaction step (2), the amount of the proteolytic enzyme to be added is, per 100 parts by mass of the flesh fractions, preferably 0.5 parts by mass or more, more preferably 1 part by mass or more, and most preferably 3 parts by mass or more. The amount of the proteolytic enzyme to be added is also, per 100 parts by mass of the flesh fractions, preferably 20 parts by mass or less, more preferably 15 parts by mass or less, and most preferably 10 parts by mass or less. The amount of 0.5 parts by mass or more of the proteolytic enzyme being added per 100 parts by mass of the flesh fractions favorably promotes the enzyme reaction, and the amount of 20 parts by mass or less of the proteolytic enzyme being added per 100 parts by mass of the flesh fractions is preferable in terms of both the amount of the active ingredient to be obtained and cost.

Prior to the addition of the proteolytic enzyme, it is preferable to add water to the flesh fractions. The amount of the water to be added to the flesh fractions is, per 100 parts by mass of the flesh fractions, preferably 50 parts by mass or more, more preferably 100 parts by mass or more, and most preferably 150 parts by mass or more. The amount of the water to be added to the flesh fractions is also, per 100 parts by mass of the flesh fractions, preferably 500 parts by mass or less, more preferably 300 parts by mass or less, and most preferably 250 parts by mass or less. The amount of 50 parts by mass or more of the water being added per 100 parts by mass of the flesh fractions promotes uniform diffusion of the proteolytic enzyme with respect to the flesh fractions, thereby favorably promoting the enzyme reaction. On the other hand, the amount of 500 parts by mass or less of the water being added per 100 parts by mass of the flesh fractions optimizes the concentration of the proteolytic enzyme in a mixture of the flesh frictions and the water, thereby favorably promoting the enzyme reaction.

The flesh fractions (or the mixture of the flesh frictions and the water) prior to the addition of the proteolytic enzyme preferably has a pH in the range from 4.5 to 6.0. The pH of the flesh fractions (or the mixture of the flesh frictions and the water) in the above range favorably promotes the enzyme reaction.

A reaction temperature in the enzyme reaction is preferably 40° C. or more and more preferably 45° C. or more. The reaction temperature in the enzyme reaction is also preferably 65° C. or less. The reaction temperature in the enzyme reaction in the above range favorably promotes the enzyme reaction.

A duration of the enzyme reaction is preferably 1 hour or more, more preferably 3 hours or more, and most preferably 4 hours or more. The duration of the enzyme reaction is also preferably 20 hours or less. The reaction duration of 20 hours or less in the enzyme reaction satisfactorily prevents decomposition of serotonin contained in the banana-derived composition to be produced.

Furthermore, the enzyme reaction step (2) preferably includes: the first enzyme reaction step performed at a reaction temperature of 45° C. or more to 55° C. or less for a reaction time in the range from 3 hours to 5 hours; and a second enzyme reaction step performed after the first enzyme reaction step at a reaction temperature of above 55° C. to 65° C. or less for a reaction time in the range from 1 hours to 17 hours. When the enzyme reaction step includes the first enzyme reaction step and the second enzyme reaction step as described above, the enzyme reaction is favorably promoted.

### [Enzyme Deactivation Step (3)]

Subsequently, the proteolytic enzyme contained in the enzymatic reactant resulting from the aforementioned enzyme reaction is deactivated. Although a method for deactivating the proteolytic enzyme contained in the enzymatic reactant is not particularly limited, for example, a method of deactivation by heating or a method of deactivation with alcohol having a carbon number of 1 to 4 is possible. The above method of deactivation may be combined as appropriate.

### (Deactivation by Heating)

Hereinafter, a description is given of a method for obtaining the composition A by deactivating the proteolytic enzyme by heating. The deactivation by heating requires only a simple operation and a simple facility, and by the above method, a high production efficiency of the composition A is achieved. In detail, the enzymatic reactant resulting from the aforementioned enzyme reaction is filtrated as needed before being heated. Although a temperature (a product temperature of the enzymatic reactant) at the time of heating is not particularly limited as long as the temperature allows the deactivation of the proteolytic enzyme, the temperature is preferably from 85° C. or more to 95° C. or less. The temperature of 85° C. or more at the time of heating satisfactorily promotes the deactivation, and the temperature of 95° C. or less at the time of heating reduces an adverse effect caused by thermal decomposition or the like of the active ingredient. Furthermore, although a heating time is not particularly limited, the heating time of 10 minutes or more and 60 minutes or less is preferable. With heating time in the above range, the deactivation is satisfactorily completed.

Subsequent to the deactivation by heating, water is removed as needed to obtain the composition A (the primary refined product). At this time, when water is removed until the composition A is turned to a dried solid (having a solid content concentration of, for example, 90% by mass or more), the composition A forms the banana-derived composition that exhibits the advantageous effect according to the present invention. The composition A exhibits excellent actions of inhibiting the blood sugar level rise and promoting collagen production. When water is removed until the composition A is turned to a concentrated liquid (having a solid content concentration of, for example, 10% by mass or more and less than 90% by mass), the composition A may be used as it is in the desugaring and desalting step (4) which is later described.

A method for removing the water is not particularly limited, and for example, concentration, freeze drying, and spray drying are possible. Among the above methods, when the composition A is turned to a dried solid, freeze drying is preferable because freeze drying reduces the thermal hysteresis of the banana-derived composition to be produced, resulting in improved biological activity of the composition. The above methods for removing water may be employed alone or in a combination of two or more. For example, freeze drying may be performed subsequent to the deactivation by heating that follows concentration. Alternatively, spray drying may be performed subsequent to the deactivation by heating. Conditions of the drying may be appropriately regulated. Prior to the drying, an excipient may also be added. The amount of the excipient to be added may be appropriately regulated.

### (Deactivation by Alcohol)

Hereinafter, a description is given of the method for obtaining the composition A by deactivating the proteolytic enzyme with the alcohol having the carbon number of 1 to 4. The deactivation with the alcohol produces an excellent yield of the active ingredient contained in the composition A to be produced. When the deactivation is performed with the alcohol having the carbon number of 1 to 4, the step (3) preferably includes: the step (i) (which may be abbreviated below as the “solid-liquid separation step (i)” as appropriate) of adding the alcohol having the carbon number of 1 to 4 to the enzymatic reactant resulting from the enzyme reaction, and subsequently subjecting a resulting alcohol-added substance to solid-liquid separation to extract a liquid phase; and the step (ii) (which may be abbreviated below as the “solvent removing step (ii)” as appropriate) of removing the alcohol having the carbon number of 1 to 4 from the liquid phase.

—Solid-Liquid Separation Step (i)—

The alcohol having the carbon number of 1 to 4 is added to the enzymatic reactant in order to deactivate the proteolytic enzyme. By doing so, the active ingredient is also extracted in the liquid phase containing the alcohol. Furthermore, by adding the alcohol having the carbon number of 1 to 4 to the enzymatic reactant, solid-liquid separation, in particular, filtration which is later described, is facilitated. In order to satisfactorily extract the active ingredient contained in the enzymatic reactant resulting from the enzyme reaction, it is preferable to leave, after the alcohol having the carbon number of 1 to 4 is added to the enzymatic reactant, the resulting alcohol-added substance at a temperature in the range from 10° C. to 30° C. for 2 to 20 hours before subjecting the alcohol-added substance to the solid-liquid separation.

The alcohol having the carbon number of 1 to 4 that is used in the solid-liquid separation step (i) is alcohol whose molecular structure has 1 to 4 carbon atoms and may be methanol, ethanol, 1-Propanol, 2-Propanol, 1-butanol, or the like. The above types of alcohol may be used alone or in a combination of two or more. Among the above types of alcohol, at least one of methanol and ethanol is preferable. Methanol or ethanol, which has a lower boiling point than other types of alcohol, is easily removed by evaporation. Accordingly, use of methanol and ethanol helps reduce the thermal hysteresis of the banana-derived composition to be produced, resulting in improved biological activity such as the action of inhibiting the blood sugar level rise. Furthermore, compared with other types of alcohol, methanol and ethanol have a high efficiency of extraction of the amino acid and the tryptophan derivative that are contained in the flesh part of a banana. Moreover, from the viewpoint of using the produced banana-derived composition in food, ethanol is more preferable for use.

In the solid-liquid separation step (i), the amount of the alcohol having the carbon number of 1 to 4 to be added is not particularly limited. For example, the amount of the alcohol having the carbon number of 1 to 4 to be added is, per 100 parts by mass of the flesh fractions, preferably 100 parts by mass or more, more preferably 200 parts by mass or more, and most preferably 250 parts by mass or more. The amount of the alcohol having the carbon number of 1 to 4 to be added is also, per 100 parts by mass of the flesh fractions, preferably 1500 parts by mass or less, more preferably 1200 parts by mass or less, even more preferably 400 parts by mass or less, and most preferably 350 parts by mass or less. The amount of the alcohol of 100 parts by mass or more being added per 100 parts by mass of the flesh fractions improves the efficiency of solid-liquid separation in the resulting alcohol-added substance, and the amount of the alcohol of 1500 parts by mass or less being added per 100 parts by mass of the flesh fractions facilitates the removal of the alcohol in the subsequent solvent removing step (ii).

A method for the solid-liquid separation (separating the liquid phase from a solid phase) of the alcohol-added substance resulting from adding, to the enzymatic reactant, the alcohol having the carbon number of 1 to 4 is not particularly limited as long as the method allows the liquid phase to be separated from the solid phase of the resulting alcohol-added substance and to be extracted. For example, filtration and the centrifugal separation method may be possible. Among the above methods, filtration is preferable from the viewpoint of reliable solid-liquid separation. As a method for filtrating the alcohol-added substance, for example, natural filtration, suction filtration, and pressure filtration are possible.

—Solvent Removing Step (ii)—

From the liquid phase extracted in the solid-liquid separation step (i), a part or an entirety of the alcohol having the carbon number of 1 to 4 is removed to obtain the composition A (the primary refined product). When the liquid phase contains water, a part or an entirety of the contained water may be removed at the same time. At this time, when the solvent (the alcohol having the carbon number of 1 to 4 and the water, which is optional) is removed until the composition A is turned to a dried solid (having a solid content concentration of, for example, 90% by mass or more), the composition A forms the banana-derived composition that exhibits the advantageous effect according to the present invention. The composition A exhibits the excellent actions of inhibiting the blood sugar level rise and promoting collagen production. When the solvent is removed until the composition A is turned to a concentrated liquid (having a solid content concentration of, for example, 10% by mass or more and less than 90% by mass), the composition A may be used as it is in the desugaring and desalting step (4) which is later described.

A method for removing the alcohol having the carbon number of 1 to 4 (and the water, which is optional) from the liquid phase is not particularly limited, and for example, concentration, freeze drying, and spray drying are possible. Among the above methods, when the composition A is turned to a dried solid, freeze drying is preferable because freeze drying reduces the thermal hysteresis of the banana-derived composition to be produced, resulting in improved biological activity of the composition. The above methods for removing the alcohol having the carbon number of 1 to 4 (and the water, which is optional) from the liquid phase may be used alone or in a combination of two or more. For example, the obtained liquid phase may be concentrated and subsequently, freeze dried. Alternatively, the obtained liquid phase may be concentrated and subsequently, spray dried. Prior to the drying, an excipient may also be added. The amount of the excipient to be added may be appropriately regulated.

Although conditions of the freeze drying are not particularly limited, for example, the following conditions are preferable. That is to say, firstly, the obtained liquid phase (or the liquid phase of the like after being concentrated) is pre-frozen for 1 to 3 hours until a product temperature reaches the range from −30° C. to −25° C. Subsequently, after the liquid phase is controlled under reduced pressure at a degree of vacuum of 40 kPa or less at a product temperature in the range from −30° C. to −25° C., a shelf temperature is gradually increased to approximately 40° C., and then, after 3 or more hours elapses since the temperature of the obtained liquid phase (or the liquid phase or the like after being concentrated) reaches a temperature substantially equal to the shelf temperature, the drying is ended.

### [Desugaring and Desalting Step (4)]

The above method of producing a banana-derived composition according to the present invention may further include the step, performed after the above steps, of desugaring and desalting the obtained composition A to obtain the composition B. By desugaring and desalting the composition A, a part or an entirety of a sugar content, salt (mineral salt and organic salt), and a free metal ion is removed, and the resulting composition B (the secondary refined product) exhibits the excellent action of antioxidation in addition to excellent actions of inhibiting the blood sugar level rise and promoting collagen production.

In the present invention, the desugaring and desalting step may be performed by a desugaring process and a desalting process which are independent from each other or may be performed by a single process.

Example of a method for desugaring and desalting the composition A (the method for performing the desugaring and desalting step by a single process at the same time) include a method (which may be abbreviated below as an “adsorptive resin method” as appropriate) of making an ingredient contained in the composition A adsorbed to an adsorptive resin filled in a column, rinsing the adsorptive resin, and subsequently eluting, from the adsorptive resin, the ingredient adsorbed to the adsorptive resin with an eluting solution. From the viewpoint of simplifying the step, the desugaring and desalting step is preferably performed by a single process, and the adsorptive resin method is more preferably adopted.

An example adopting the adsorptive resin method in the desugaring and desalting step (4) is described in detail below.

The adsorptive resin method preferably includes: the step (the water addition step) of adding water to the composition A as needed; the step (the adsorption step) of passing the composition A (or a composition obtained by adding the water to the composition A, i.e., a water-containing composition) though the column filled with the adsorptive resin to make an ingredient (e.g. an amino acid) contained in the composition A adsorbed to the adsorptive resin; the step (the rinse step) of passing water through the column filled with the adsorptive resin for purification; the step (the elution step) of eluting the ingredient adsorbed to the adsorptive resin by passing the eluting solution through the column filled with the adsorptive resin, to obtain an eluted fraction by the eluting solution; and the step (the eluting solution removing step) of removing the eluting solution from the eluted fraction. The following describes the above steps in detail.

### (Water Addition Step)

Water is added to the composition A as needed. For example, when the composition A is a dried solid, it is necessary to add water to turn the composition A to a solution. When the composition A is a concentrated liquid including a solvent, the concentrated liquid may be used as it is, or it is also possible to add water for regulation of the solid content concentration. Although the solid content concentration of the composition A (or the water-containing composition thereof) before passing through the column filled with the adsorptive resin is not particularly limited, the solid content concentration in the range from 3% by mass to 30% by mass is preferable.

### (Adsorption Step)

The composition A (or the water-containing composition thereof) is passed though the column filled with the adsorptive resin to make an ingredient (e.g. an amino acid) contained in the composition A adsorbed to the adsorptive resin. As the adsorptive resin, for example, a synthetic adsorbent that adsorbs an organic substance in the solution by a physical interaction between a pore surface of the resin and a substance to be adsorbed or a cation exchange resin with a sulfonic group may be used. Among the above examples, the cation exchange resin is preferable because the cation exchange resin has an efficient adsorption capability of the amino acid such as phenylalanine and tryptophan and the tryptophan derivative such as serotonin, and a strongly acidic cation exchange resin is more preferable. Examples of the strongly acidic cation exchange resin include DIAION™ SK1B (manufactured by Mitsubishi Chemical Industries Ltd.) and Amberlite™ (manufactured by Dow Chemical Company).

A flow rate of the composition A (or the water-containing composition thereof) that is passed through the column filled with the adsorptive resin may be appropriately determined in accordance with properties and condition of the composition A (or the water-containing composition thereof) to be passed through and the adsorptive resin that is used. Although the flow rate is not particularly limited, the composition A (or the water-containing composition thereof) may be passed through at a rate of flow in the range from 0.5 SV to 5 SV. The unit SV refers to a quantity (a volume-based quantity measured at a temperature of 25° C.) of a solution that is passed through the column with respect to the adsorptive resin per unit time, and when the same volume of the solution as the volume of the adsorptive resin is passed through the column in 1 hour, the flow rate of the solution is defined as 1 SV.

Furthermore, a flow volume of the composition A that is passed through the column filled with the adsorptive resin is not particularly limited, and the flow volume may be in the range from, for example, 1 RV to 30 RV. The unit RV refers to a quantity (a volume-based quantity measured at the temperature of 25° C.) of a solution that is passed through the column with respect to the adsorptive resin, and when the same volume of the solution as the volume of the adsorptive resin is passed through the column, the flow volume of the solution is defined as 1 RV.

### (Rinse Step)

Subsequently, water is passed through the above column filled with the adsorptive resin. By passing water through the column after the aforementioned suction step, a non-adsorbed ingredient and an ingredient that is difficult to be adsorbed (e.g. a saccharide, organic salt, mineral salt, and a free metal ion) is removed. That is to say, desugaring and desalting are performed.

A flow rate of the water that is passed through the column after the adsorption step may be appropriately determined in accordance with the adsorptive resin that is used. Although the flow rate is not particularly limited, the water may be passed through the column at a flow rate in the range from 0.5 SV to 5 SV. Passing the water through the column at a flow rate in the above range allows effective desugaring and desalting.

Furthermore, a flow volume of the water that is passed through the column after the adsorption step may be appropriately determined in accordance with the adsorptive resin that is used. Although the flow volume is not particularly limited, the flow volume is in the range from, for example, 1 RV to 30 RV. Passing the water of a volume in the above range through the column allows satisfactory desugaring and desalting.

### (Elution Step)

Subsequently, an eluting solution is passed through the column filled with the adsorptive resin. By doing so, an ingredient adsorbed to the adsorptive resin is eluted, and a fraction eluted by the eluting solution is obtained. The eluting solution (which is also referred to as an eluate) is a liquid used for eluting, from the adsorptive resin, the ingredient adsorbed to the adsorptive resin. The eluting solution to be used is not particularly limited as long as the eluting solution has the above function. As an example, when the synthetic adsorbent such as SEPABEADS™ SP207 is used as the adsorptive resin, a mixed solvent of water and alcohol (methanol and/or ethanol) may be used as the eluting solution. Although a concentration of the methanol and/or the ethanol contained in the mixed solvent is not particularly limited, the methanol and/or the ethanol preferably has a concentration in the range from 3 to 10% by mass. The concentration of the methanol and/or the ethanol contained in the mixed solvent in the above range allows efficient elution of the ingredient adsorbed to the adsorptive resin. As another example, when the strongly acidic cation exchange resin such as DIAION™ SK1B is used as the adsorptive resin, ammonia water may be used as the eluting solution. Although an ammonia concentration of the ammonia water is not particularly limited, an ammonia concentration in the range from 0.1 mol/L to 5 mol/L is preferable. The ammonia concentration of the ammonia water in the above range allows efficient elution of the ingredient adsorbed to the adsorptive resin.

A flow rate of the eluting solution that is passed through the column in the elution step may be appropriately determined in accordance with the absorbing resin that is used. Although the flow rate is not particularly limited, the eluting solution is passed through the column at a flow rate in the range, for example, from 0.5 SV to 5 SV. Passing the water through the column at a flow rate in the above range allows efficient elution of the ingredient adsorbed to the adsorptive resin.

Furthermore, a flow volume of the eluting solution that is passed through the column in the elution step may be appropriately determined in accordance with the adsorptive resin that is used. Although the flow volume is not particularly limited, the flow volume is in the range from, for example, 5 RV to 60 RV. Passing the eluting solution of a volume in the above range through the column allows more reliable elution of the ingredient adsorbed to the adsorptive resin.

### (Eluting Solution Removing Step)

From the eluted fraction obtained in the elution step, the eluting solution is removed so as to obtain the composition B (a dried solid: the secondary refined product). A method for removing the eluting solution so as to obtain the composition B in the form of a dried solid is not particularly limited, and the methods described in the section “Solvent Removing Step (ii)” above may be adopted. Among the above methods, freeze drying is preferable because freeze drying reduces the thermal hysteresis of the banana-derived composition to be produced, resulting in improved biological activity of the composition. Furthermore, as in “Solvent Removing Step (ii)”, the above methods for removing the eluting solution may be used alone or in a combination of two or more. For example, the obtained eluted fraction may be concentrated and subsequently, freeze dried. Alternatively, the obtained eluted fraction may be concentrated and subsequently, spray dried. Conditions of the freeze drying are not particularly limited, and the conditions described in the section “Solvent Removing Step (ii)” are preferably adopted. The obtained composition B may be used as the banana-derived composition, and the composition B exhibits the excellent actions of inhibiting the blood sugar level rise, promoting collagen production, and antioxidation.

### <Biologically Active Substance>

The biologically active substance according to the present invention is characterized by including, as the active ingredient, a banana-derived composition obtained from a flesh part of the banana. The biologically active substance according to the present invention exhibits excellent biological activity such as the actions of inhibiting the blood sugar level rise and promoting collagen production.

### [Banana-Derived Composition]

The banana-derived composition that the biologically active substance according to the present invention includes as the active ingredient is not particularly limited as long as the composition is obtained from a flesh part of a banana. The banana-derived composition is preferably obtained from the flesh part of an unripe banana for substantially the same reasons as those described in the section “Method of Producing Banana-Derived Composition” above.

A method for obtaining the banana-derived composition that the biologically active substance according to the present invention includes as the active ingredient is not particularly limited. For example, when the banana-derived composition is obtained through extraction, methods of extraction such as using hot water, fluid carbon dioxide, and alcohol may be employed.

It is preferable that the banana-derived composition is obtained by adding a proteolytic enzyme to the flesh part of the banana for decomposing proteins contained in the flesh part of the banana through the enzyme reaction, and subsequently, by deactivating the proteolytic enzyme by heating or with alcohol. As the alcohol, alcohol having the carbon number of 1 to 4 is preferably used, and as the alcohol having the carbon number of 1 to 4, those described in “Method of Producing Banana-Derived Composition” above may be used. Preferable types of the alcohol having the carbon number of 1 to 4 are also substantially the same as those described in “Method of Producing Banana-Derived Composition” above.

Furthermore, it is preferable that the banana-derived composition that the biologically active substance according to the present invention includes as the active ingredients is produced by the method of producing a banana-derived composition according to the present invention. For example, when the banana-derived composition is produced by the aforementioned method of producing a banana-derived composition according to the present invention, the biologically active substance including the composition A (the primary refined product) exhibits the excellent actions of inhibiting the blood sugar level rise and promoting collagen production. Furthermore, the biologically active substance including the composition B (the secondary refined product) that has undergone the desugaring and desalting process exhibits the excellent actions of inhibiting the blood sugar level rise, promoting collagen production, and antioxidation.

Moreover, in the biologically active substance according to the present invention, it is preferable that the banana-derived composition contains an amino acid and a tryptophan derivative, and a total amount of the amino acid and the tryptophan derivative is preferably 7% by mass or more, more preferably 8% by mass or more, and most preferably 10% by mass or more. When the total amount of the amino acid and the tryptophan derivative contained in the banana-derived composition is 7% by mass or more, the biological activity of the biologically active substance according to the present invention is improved. For example, when the method according to the present inventions is used to produce the banana-derived composition, the amount of the amino acid and the tryptophan derivative may be increased, for example, by regulating the conditions of the enzyme reaction or by performing the desugaring and the desalting step (4). Although an upper limit of the total amount of the amino acid and the tryptophan derivative contained in the banana-derived composition is not particularly limited, the total amount of the amino acid and the tryptophan derivative is typically 95% by mass or less.

Moreover, in the banana-derived composition, it is preferable that the amino acid includes phenylalanine and tryptophan and that the tryptophan derivative includes serotonin. When the banana-derived composition includes phenylalanine, tryptophan, and serotonin, the biological activity of the biologically active substance according to the present invention is improved.

Additionally, in the present invention, the amounts of the amino acid and the tryptophan derivative, and the amounts of phenylalanine, tryptophan, and serotonin that are contained in the banana-derived composition may be measured with use of measurement methods described in Examples described herein.

The biologically active substance according to the present invention may include an ingredient, such as an excipient, other than a banana-derived composition depending on the application purpose. Furthermore, the application purpose is not particularly limited, and the biologically active substance may be blended in various foods and drinks or may be used alone or in a combination with another ingredient as a supplement.

### EXAMPLES

The present invention will be described in detail below in accordance with Examples. However, the present invention is not limited to the Examples. In the description below, “%” and “parts” represent a mass-based amount unless otherwise defined.

For measurement of the Brix degree and ingredient analysis of the banana-derived compositions, the following methods were used.

### <Measurement of Brix Degree>

The Brix degree was measured by means of a sugar refractometer (manufactured Koshimizu: a sugar scale hydrometer WZ-113) at a measurement temperature of 25° C.

### <Ingredient Analysis of Banana-Derived Composition>

The content and the type of the amino acid were measured by means of amino acid automatic analyzers (manufactured by JEOL Ltd. and Hitachi High-Technologies Corporation). The content and the type of the tryptophan derivative (serotonin) were measured by means of a high-performance liquid chromatography (manufactured by Shimadzu Corporation) by using a reagent of the corresponding compound according to the absolute calibration method. Then, the content and the type of sugar were measured by means of Shodex RI-71 (manufactured by Showa Denko K.K.) by using a reagent of a presumed sugar) according to the absolute calibration method.

### Example 1

### Production of Banana-Derived Composition 1 (Composition A: Primary Refined Product, which is Deactivated with Alcohol)

From 196.5 parts of a fruit of an unripe banana (variety: the Giant Cavendish variety, Brix degree: 1.4° Bx), a peel was pulled off to obtain 104.2 parts of a flesh part. The obtained flesh part was fractioned by means of a cutter mill crusher into 100 parts of pasty flesh fractions. Into the obtained 100 parts of flesh fractions, 190 parts of ion exchanged water was added and stirred, and moreover, as the proteolytic enzyme, 5 parts of Protease M “Amano” SD was added to obtain a mixture. The mixture was heated to a temperature of 50° C., and this temperature was maintained for 4 hours. Subsequently, the mixture was further heated to a temperature of 58° C. to 60° C., and this temperature was maintained for 2 hours for promoting the enzyme reaction. Thus obtained enzymatic reactant (295 parts) was left to be cooled down to a temperature of 25° C. to 30° C., and into the cooled enzymatic reactant, 291.1 parts of ethanol was added and stirred at a temperature of 25° C. to 30° C. for 2 hours. Thus, an alcohol (ethanol)-added substance was obtained. The obtained alcohol-added substance was filtrated at a room temperature by means of a single-plate filter. At this time, an extract ingredient remaining in the solid phase was also washed with use of 29.5 parts ethanol. With the filtration, the alcohol-added substance was separated into the solid phase (42.8 parts in light brown color) and the liquid phase (572.8 parts of a transparent liquid in yellow color). The liquid phase was condensed under vacuum while a product temperature was maintained at 40° C. or less to obtain 50 parts of the concentrated liquid. The obtained concentrated liquid was pre-frozen for approximately 2 hours until a product temperature reached a temperature of −30° C. to −25° C. Subsequently, the pre-frozen concentrated liquid was controlled under reduced pressure at a degree of vacuum of 40 kPa or less while a product temperature was maintained at from −30° C. to −25° C., and then, the shelf temperature was gradually heated to 40° C. Then, after 3 or more hours elapsed since a product temperature reached a temperature substantially equal to the shelf temperature, the freeze drying was ended. Thus, 11 parts of the banana-derived composition 1 was obtained (at a yield of 11% with respect to the flesh fractions). The obtained banana-derived composition 1 contained 5.4% of fructose, 45.4% of glucose, 0% of sucrose, 11.0% of an amino acid (0.89% of phenylalanine and 0.34% of tryptophan), and 0.004% of serotonin (tryptophan derivative).

### Example 2

### Production of Banana-Derived Composition 2 (Composition A: Primary Refined Product, which is Deactivated with Alcohol)

From 196.5 parts of a fruit of an unripe banana (variety: the Giant Cavendish variety, Brix degree: 1.4° Bx), a peel was pulled off to obtain 103.2 parts of a flesh part. The obtained flesh part was fractioned by means of a cutter mill crusher into 100 parts of pasty flesh fractions. Into the obtained 100 parts of flesh fractions, 200 parts of ion exchanged water was added and stirred, and moreover, as the proteolytic enzyme, 5 parts of Protease M “Amano” SD was added to obtain a mixture. The mixture was heated to a temperature of 50° C., and this temperature was maintained for 4 hours. Subsequently, the mixture was further heated to a temperature of 60° C., and this temperature was maintained for 2 hours for promoting the enzyme reaction. Thus obtained enzymatic reactant (305 parts) was left to be cooled down to a temperature of 23° C., and into the cooled enzymatic reactant, 1087.7 parts of ethanol was added and stirred at a temperature of 13° C. to 23° C. for 2 hours. Thus, an alcohol (ethanol)-added substance was obtained. The obtained alcohol-added substance was filtrated at the room temperature by means of the single-plate filter. At this time, the solid phase was washed with use of 14 parts ethanol for washing an extract ingredient remaining in the solid phase. With the filtration, the alcohol-added substance was separated into the solid phase (42.1 parts) and the liquid phase (1434.4 parts of a transparent liquid in orange color, as a result of further adding thereto 70.2 parts of methanol after the filtration). The liquid phase was condensed under vacuum for 11 hours while a product temperature was maintained at 40° C., and moreover, the condensed liquid phase was dried at a temperature of 40° C. for 24 hours in a tray vacuum dryer. Thus, 8.2 parts of the banana-derived composition 2 was obtained (at a yield of 8.2% with respect to the flesh fractions). The obtained banana-derived composition 2 contained 4.70% of fructose, 43.41% of glucose, 1.34% of sucrose, 13.64% of an amino acid (1.13% of phenylalanine and 0.42% of tryptophan), and 0.0009% of serotonin (tryptophan derivative).

### Example 3

### Production of Banana-Derived Composition 3 (Composition B: Secondary Refined Product, which is Deactivated with Alcohol)

In accordance with the procedure described with respect to “(Example 1) Production of Banana-Derived Composition 1” above, a flesh part of an unripe banana was fractioned and subjected to the enzyme reaction, the filtration, and the concentration. Thus, 50 kg of the concentrated liquid (including 11.0 kg of a solid content) was obtained. Into the obtained concentrated liquid, 150 kg of ion exchanged water was added to obtain 200 kg of a dilution water (with a solid content concentration of 5.5%). The obtained 200 kg of dilution water (200 L, 20 RV) was passed through a column filled with 10 L (1 RV) of the synthetic adsorbent (SEPABEADS™ SP207 manufactured by Mitsubishi Chemical Industries Ltd.) at a flow rate of 15 L/h (1.5 SV). Subsequently, 80 L (8 RV) of ion exchanged water was passed through the column at substantially the same flow rate for rinsing. Immediately before all of the ion exchanged water had been passed through, the Brix degree of the ion exchanged water that passed through the column was measured. The above Brix degree was confirmed to be zero.

Then, 180 L (18 RV) of a 5% by mass ethanol solution was passed through the column at substantially the same flow rate, and subsequently, 180 L (18 RV) of a 7% by mass ethanol solution was further passed through the column at substantially the same flow rate. A liquid (360 L) obtained after the solution was passed through was collected. Immediately before all of the 7% by mass ethanol solution had been passed through, a concentration of tryptophan contained in the liquid that passed through the column was measured by means of the high-performance liquid chromatography manufactured by Shimadzu Corporation. The above concentration was confirmed to be zero.

The 360 L of liquid that passed through the column was condensed under vacuum while a product temperature of 40° C. or less was maintained to obtain 0.83 kg of the concentrated liquid. The obtained concentrated liquid was pre-frozen for approximately 2 hours until a product temperature reached a temperature of −30° C. to −25° C. Subsequently, the pre-frozen concentrated liquid was controlled under reduced pressure at a degree of vacuum of 40 kPa or less while a product temperature was maintained at from −30° C. to −25° C., and then, the shelf temperature was gradually heated to 40° C. Then, after 3 or more hours elapsed since a product temperature reached a temperature substantially equal to the shelf temperature, the freeze drying was ended. Thus, 0.17 kg of the banana-derived composition 3 was obtained (at a yield of 1.5% with respect to the 11.0 kg of the solid content included in the initial concentrated liquid). The obtained banana-derived composition 3 contained 0% of fructose, 0% of glucose, 0% of sucrose, 3.7% of phenylalanine, 11.8% of tryptophan, and 0.15% of serotonin (tryptophan derivative).

### Example 4

### Production of Banana-Derived Composition 4 (Composition B: Secondary Refined Product, which is Deactivated with Alcohol)

In accordance with the procedure described with respect to “(Example 2) Production of Banana-Derived Composition 2” above, a flesh part of an unripe banana was fractioned and subjected to the enzyme reaction, the filtration, and the concentration. Thus, 3.5 kg of the concentrated liquid (including 0.8 kg of a solid content at the solid content concentration of 23%) was obtained. The obtained 3.5 kg (3.5 L, 3.5 RV) of the concentrated liquid was passed through a column filled with the strongly acidic cation exchange resin (DIAION™ SK1B manufactured by Mitsubishi Chemical Industries Ltd.) at a flow speed of 1.5 L/h (1.5 SV). Subsequently, 15.4 L (15.4 RV) of ion exchanged water was passed though the column at substantially the same flow rate. Immediately before all of the ion exchanged water had been passed through, the Brix degree of the ion exchanged water that passed through the column was measured. The above Brix degree was confirmed to be zero.

Then, 10 L (10 RV) of 1 mol/L ammonia water was passed through the column at substantially the same flow rate, and a liquid (10 L) obtained after the ammonia water was passed through was obtained. Immediately before all of the 1 mol/L ammonia water had been passed through, a concentration of tryptophan contained in the eluting solution that passed through the column was measured by means of the high-performance liquid chromatography manufactured by Shimadzu Corporation). The above concentration was confirmed to be zero.

The 15.4 L of the liquid that passed through the column was condensed under vacuum while a product temperature was maintained at 40° C. or less to obtain 0.70 kg of the concentrated liquid. The obtained concentrated liquid was pre-frozen for approximately 2 hours until a product temperature reached a temperature of −30° C. to −25° C. Subsequently, the pre-frozen concentrated liquid was controlled under reduced pressure at a degree of vacuum of 40 kPa or less at a product temperature in the range from −30° C. to −25° C., and then, the shelf temperature was gradually heated to 40° C. Then, after 3 or more hours elapsed since a product temperature reached a temperature substantially equal to the shelf temperature, the freeze drying was ended. Thus, 0.16 kg of the banana-derived composition 4 was obtained (at a yield of 20% with respect to 0.8 kg of the solid content included in the initial concentrated liquid). The obtained banana-derived composition 4 contained 0% of fructose, 0% of glucose, 0% of sucrose, 55.2% of an amino acid (4.47% of phenylalanine and 1.69% of tryptophan), and 0.015% of serotonin (tryptophan derivative). An amount of potassium contained in the banana-derived composition 4 was also measured. The amount of potassium was as very small as 7 mg/100 g, which showed an excellent desalting result.

### Comparative Example 1

### Production of Banana-Derived Composition 5

In accordance with the procedure described with respect to Example 1 except for that the enzymolysis was omitted, 1.63 parts of the banana-derived composition was obtained. The yield was considerably small compared with the banana-derived composition 1 according to Example 1. Accordingly, it can be understood that the enzymolysis dramatically increases the yield of a banana-derived composition. The obtained banana-derived composition 5 contained 16.3% of fructose, 25.6% of glucose, 0% of sucrose, 6.6% of amino acid (0.3% of phenylalanine and 0.102% of tryptophan), and 0.009% of serotonin (the tryptophan derivative). Thus, the amount of the active ingredient was fairly small compared with the banana-derived composition 1 according to Example 1, and use of the banana-derived composition 5 does not provide sufficient biological activity of the actions of inhibiting the blood sugar level rise and promoting collagen production.

### Example 5

### Production of Banana-Derived Composition 6 (Composition A: Primary Refined Product, which is Deactivated by Heating)

From 196.5 parts of a fruit of an unripe banana (variety: the Giant Cavendish variety, Brix degree: 1.4° Bx), a peel was pulled off, and 104.2 parts of a flesh part was obtained. The obtained flesh part was fractioned by means of a cutter mill crusher into 100 parts of pasty flesh fractions. Into the obtained 100 parts of flesh fractions, 190 parts of ion exchanged water was added and stirred, and moreover, as the proteolytic enzyme, 5 parts of Protease M “Amano” SD was added to obtain a mixture. The obtained mixture was heated to a temperature of 50° C., and this temperature was maintained for 4 hours. Subsequently, the mixture was further heated to a temperature of 60° C., and this temperature was maintained for 15 hours for promoting the enzyme reaction. Thus obtained enzymatic reactant (295 parts) was subjected to celite filtration and further filtrated with use of a filter having a pore size of 0.45 μm. A filtrate obtained was heated at a temperature of 90° C. for 30 minutes for deactivating the proteolytic enzyme, and subsequently, the filtrate was left to be cooled down to a temperature of 25° C. to 30° C. Thus, an enzymatic reactant was obtained. The obtained enzymatic reactant was condensed under vacuum while a product temperature was maintained at 40° C. or less to obtain the solid content concentration of approximately 10% by mass. Into the enzymatic reactant that was condensed under vacuum, as the excipient, the same amount of Pinedex™ #100 as the amount by mass of the solid content included in the enzymatic reactant was added and sterilized by stirring at a temperature of 90° C. for 1 hour. After the sterilization by stirring, the enzymatic reactant was left to be cooled down to a temperature of 25° C. to 30° C. Thus, 16.8 parts of the banana-derived composition 6 was obtained.

### <Confirmation of Action of Inhibiting Blood Sugar Level Rise (in Rats)>

After 24 hours of fasting, a male Wistar rat (N=5) of age 7 weeks was administered 10 mg of the banana-derived composition 2 (primary refined product) as the biologically active substance, mixed with 2 mL of water per 1 g of rat body weight. The mixture of the banana-derived composition 2 and water was directly administered into the stomach of the rat (N=5) with use of a stomach tube. Blood was collected from a caudal vein of the rat before the administration of the banana-derived composition 2, and after 15 minutes, 30 minutes, 45 minutes, and 60 minutes from the administration, and for each of the collected blood, the blood sugar level was measured by means of Medisafe Fit manufactured by Terumo Corporation. All the above operations were performed under anesthesia (it was additionally confirmed that the anesthesia did not have any influence on the blood sugar level).

A week later, the same rat (N=5) was similarly administered, as a placebo instead of the banana-derived composition 2, a sugar content dissolved in water. The above sugar content was the same type and administered in the same amount as the sugar content (fructose and glucose) contained in the banana-derived composition 2. Blood was collected from a caudal vein of the rat before the administration of the placebo, and after 15 minutes, 30 minutes, 45 minutes, and 60 minutes from the administration, and for each of the collected blood, the blood sugar level was measured. All the above operations were performed under anesthesia.

Furthermore, another male Wistar rat (N=5) of age 7 weeks was tested with substantially the same placebo as above, and the blood sugar level was measured in a manner similar to the above. A week later, in a manner similar to the above, the rat was tested with the banana-derived composition 2, and the blood sugar level was measured. FIG. 1 shows changes in blood sugar level over time, for the case where the banana-derived composition 2 was administered and the case where the placebo was administered (with respect to each of the cases, the mean blood sugar level of N=10 is shown). From FIG. 1, it can be seen that the banana-derived composition 2 (the primary refined product) as the biologically active substance exhibits the excellent action of inhibiting the blood sugar level rise.

### <Confirmation of Action of Inhibiting Blood Sugar Level Rise (in Human)>

A test subject (a healthy Japanese male of age above 20 years and below 70 years) had supper by 21 o'clock on a day before a test. Subsequently, the test subject was put on fasting (only an intake of water was allowed), followed by total fasting performed from 1 hour before the time (approximately 9 o'clock a.m.) for an intake of the sample (a placebo, which is the banana-derived composition 6) to the time when collection of blood was all completed on the day of the test.

Firstly, the test subject was tested with the placebo. The test subject ingested 12.8 g of the placebo (a mixture specially prepared by mixing glucose, fructose, and maltitol in the same amount as the amount of glucose, fructose, and maltitol as the excipient contained in 15 g of the banana-derived composition 6) together with 200 mL of water. Subsequently, the test subject ingested a load diet (300 g of cooked rice, Kukure Curry™ medium hot) by ingesting one-third of the load diet three times, each time for approximately 5 minutes, and the whole of the load diet in 15 minutes in total. Blood was collected after the intake of the placebo, before the ingestion of the load diet, and after 30 minutes, 60 minutes, and 90 minutes from when the test subject finished ingesting the load diet, for measurement of the blood sugar level and insulin.

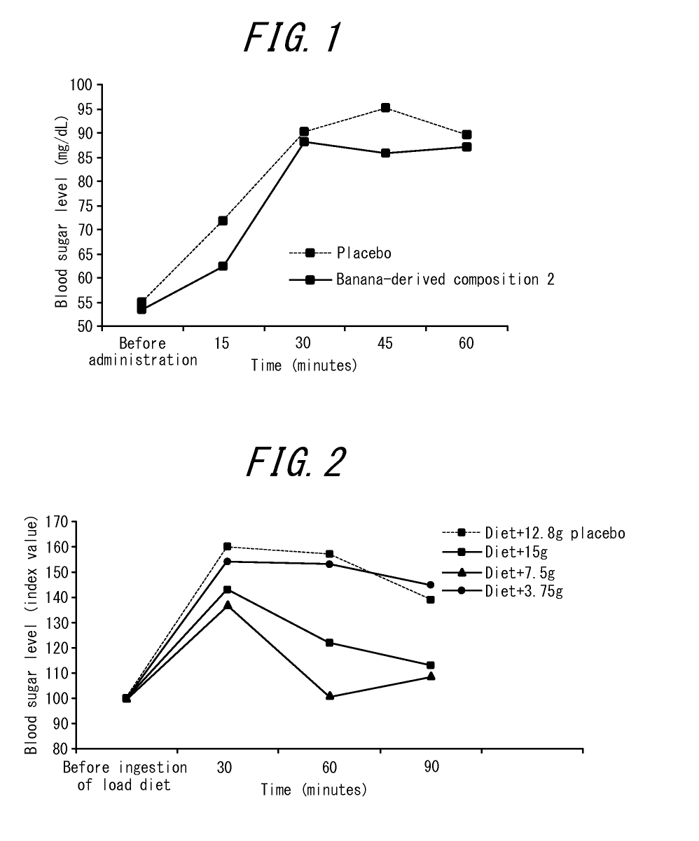

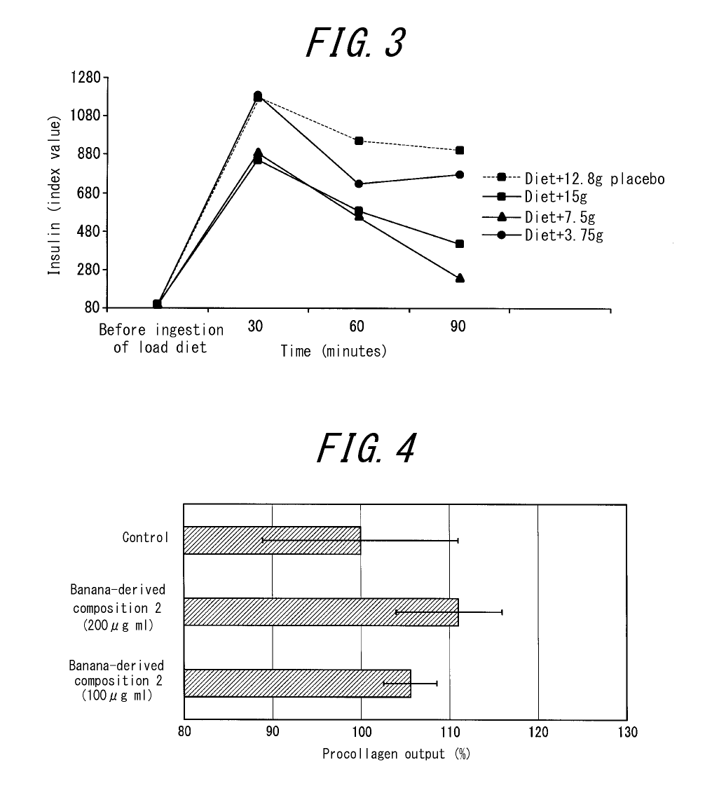

Subsequently, after an interval of 4 days from a day on which the above test with the placebo was performed, the test subject was tested with the banana-derived composition 6. The test subject ingested 15 g of the banana-derived composition 6 together with 200 mL of water, and subsequently, the test subject ingested the aforementioned load diet by ingesting one-third of the load diet three times, each time for approximately 5 minutes, and the whole of the load diet in 15 minutes in total Blood was collected after the intake of the banana-derived composition 6, before the ingestion of the load diet, and after 30 minutes, 60 minutes, and 90 minutes from when the test subject finished ingesting the load diet, for measurement of the blood sugar level and insulin. After an interval of 2 days after that, operations substantially the same as the above except for that the amount of the banana-derived composition 6 was 7.5 g were performed, and after another interval of 2 days, operations substantially the same as the above except for that the amount of the banana-derived composition 6 was 3.75 g were performed. FIGS. 2 and 3 respectively show changes in blood sugar level and insulin over time represented by index values (where a value before the ingestion of the load diet is indexed as 100), for the case where the banana-derived composition 6 was administered and the case where the placebo was administered. From FIGS. 2 and 3, the banana-derived composition 6 (the primary refined product) as the biologically active substance exhibits the excellent action of inhibiting the blood sugar level rise.

### <Confirmation of Action of Promoting Collagen Production>

A normal human dermis fibroblast was seeded in a 24 well culture plate at a concentration of 2×10⁴ cells/ml and placed in a CO₂ incubator at 37° C. for 72 hours. As a medium, DMEM medium (manufactured by SIGMA) supplemented with 5% fetal bovine serum (manufactured by Thermo Trace Ltd.) was used. After the medium was removed, an authentic culture medium was formed in the 24 well culture plate in an amount of 1 mL per unit well. As the culture medium, DMEM medium (manufactured by SIGMA) supplemented with 0.25% fetal bovine serum (manufactured by Thermo Trace Ltd.) was used. After a lapse of 24 hours from when the medium was replaced with the culture medium, the culture medium was further replaced with another culture medium that was substantially the same as the previous one. Then, samples were added to the culture medium. As the samples, a control (distilled water), the banana-derived composition 2 (at an additive concentration of 100 μg/ml), and the banana-derived composition 2 (at an additive concentration of 200 μg/ml) were used (the test samples were prepared by using distilled water and subjected to filtration sterilization with a 0.22 μm filter).

After the samples were added, the samples were incubated in the CO₂ incubator at 37° C. for 72 hours, followed by measurement of a procollagen output after the incubation. In detail, an output of type I procollagen found in a supernatant liquid of the culture was measured by means of Procollagen Type I C-Peptide (RIP) EIA Kit (manufactured by Takara Bio, Inc.), which is capable of determining Procollagen Type I C-Peptide (PIP). The number of cells was also determined by means of Cell counting Kit-8 (manufactured by Dojin Chemical Co., Ltd.) FIG. 4 shows the procollagen output, for the case where the control was added and for the case where the banana-derived composition 2 was added (with respect to each of the cases, a mean value of N=4 is shown, and an error bar indicates an actual range of a measurement result, where the output of the control is defined as 100%). From FIG. 4, it can be seen that the banana-derived composition 2 (the primary refined product) as the biologically active substance exhibits the excellent action of promoting collagen production.

### <Confirmation of Action of Antioxidation>

The banana-derived composition 4 (the secondary refined product) as the biologically active substance and vitamin C were dissolved in ultrapure water to prepare solutions each at a concentration of 10 mg/ml. An antioxidation ability was measured twice for each of the prepared solutions. In detail, the antioxidation ability against oxidation through hypochlorous acid (HClO) was measured by means of Free Radical Elective Evaluator (FREE) manufactured by Wismerll Co., Ltd. Oxygen (OXY) Adsorbent Test was applied to the measurement. As a result of the measurement, a concentration of HClO neutralized is obtained in the unit of μmol HClO/mL. A higher concentration value means a better HClO neutralization ability, that is to say, a stronger antioxidative action. Table 1 shows the result.

TABLE 1

| | Measurement result (μmol HClO/mL) | | |
|---|---|---|---|
| | 1st measurement | 2nd measurement | Average |
| Banana-derived | 199.1 | 182.2 | 190.7 |
| composition 4 | | | |
| Vitamin C | 71.7 | 79.7 | 75.7 |

From Table 1, it can be seen that the banana-derived composition 4 (the secondary refined product) as the biologically active substance yields a higher μmol HClO/mL value, that is to say, exhibits a stronger action of antioxidation.

### <Confirmation of Filterability (Solid-Liquid Separation)>

In order to confirm filterability for a case where a flesh part of an unripe banana (variety: the Giant Cavendish variety, Brix degree: 4.5° Bx) was used as a material and for a case where a flesh part of a ripe banana (variety: the Giant Cavendish variety, Brix degree: 24.8° Bx) was used as a material, a comparative test was conducted in accordance with the following procedure. In accordance with a procedure that is substantially the same as that of the method described with respect to “(Example 1) Production of Banana-Derived Composition 1” above, the flesh part of the aforementioned unripe banana was fractioned to obtain flesh fractions (150 g). The obtained flesh fractions (150 g) were subjected to the enzyme reaction. Thus, an enzymatic decomposition product was obtained. The obtained enzymatic decomposition product was named Sample 1 of the filterability test. Samples 2 to 4 of the filterability test were also prepared by adding, into the Sample 1 of the filterability test, ethanol such that ethanol concentrations of 10%, 30%, and 50% were achieved, respectively.

On the other hand, in accordance with a procedure that is substantially the same as that of the case of the unripe banana, the flesh part of the aforementioned ripe banana was fractioned to obtain flesh fractions (150 g). The obtained flesh fractions (150 g) were subjected to the enzyme reaction. Thus, an enzymatic decomposition product was obtained. The obtained enzymatic decomposition product was named Sample 5 of the filterability test. Samples 6 and 7 of the filterability test were also prepared by adding, into the Sample 5 of the filterability test, ethanol such that ethanol concentrations of 10% and 50% were achieved, respectively.

The Samples 1 to 7 were subjected to suction filtration by means of Qualitative Filter Paper No. 2 (manufactured by ADVANTEC Co., Ltd.). By visually observing time elapsed before the filtration of whole amounts of Samples 1 to 7 was ended, time (filtration time) required for the filtration of each of the entire Samples 1 to 7 was measured. Table 2 shows a result of the measurement.

TABLE 2

| Sample | | Ethanol concentration | |
|---|---|---|---|
| No. | Banana used | (% by mass) | Filtration time |
| 1 | Unripe | 0 | 3 minutes 30 seconds |
| 2 | banana | 10 | 3 minutes 15 seconds |
| 3 | | 30 | 3 minutes 03 seconds |
| 4 | | 50 | 2 minutes 50 seconds |
| 5 | Ripe banana | 0 | 26 minutes 00 second |
| 6 | | 10 | 30 minutes 00 second |
| 7 | | 50 | 34 minutes 00 second |

From Table 2, it can be seen that the case using the flesh part of the unripe banana yields a strikingly higher filterability compared with the case using the flesh part of the ripe banana. Furthermore, it can be seen that the case using the flesh part of the unripe banana improves filterability to the extent where the ethanol concentration is increased.

### <Confirmation of Extraction Efficiency Depending on Difference in Alcohol Used for Deactivation>

In order to confirm a difference in efficiency of extraction of the amino acid and the tryptophan derivative depending on a difference in the alcohol having the carbon number of 1 to 4 that is used for deactivation of the proteolytic enzyme, a comparative test was conducted in accordance with the following procedure. The banana-derived composition 1 according to Example 1 in which ethanol was used, and three banana-derived compositions, in each of which a different one of methanol, 2-propanol, and 1-butanol was used instead of ethanol used in Example 1, were prepared. The amounts of phenylalanine, tryptophan, and serotonin contained in the above compositions were measured. Table 3 shows a result of the measurement.

TABLE 3

| Alcohol used | | Ethanol | Methanol | 2-propanol | 1-butanol |
|---|---|---|---|---|---|
| Amount | Phenylalanine | 96.53 | 97.13 | 79.3 | 45.27 |
| (mg) | Tryptophan | 35.2 | 34.71 | 35.1 | 16.71 |
| | Serotonin | 0.46 | 0.46 | 0.32 | 0.66 |

From Table 3, it can be seen that ethanol and methanol are capable of efficiently extracting the aforementioned ingredients contained in the flesh part of a banana as a whole compared with 2-propanol and 1-butanol.

According to the present invention, a method of producing a banana-derived composition by which a composition exhibiting excellent biological activity is obtained from a flesh part of a banana is provided. Furthermore, according to the present invention, a biologically active substance containing a banana-derived composition obtained from a banana flesh that exhibits favorable biological activity is provided.

1. A method of producing a banana-derived composition, the method comprising the steps of:
(1) mincing a flesh part of a banana to obtain flesh fractions;
(2) adding a proteolytic enzyme to the flesh fractions and decomposing proteins contained in the flesh fractions through an enzyme reaction to obtain an enzymatic reactant; and
(3) deactivating the proteolytic enzyme contained in the enzymatic reactant to obtain a composition A.

2. The method of producing a banana-derived composition according to claim 1, wherein
the flesh part of the banana comprises a flesh part of an unripe banana.

3. The method of producing a banana-derived composition according to claim 1, wherein
the step (3) deactivates the proteolytic enzyme by heating.

4. The method of producing a banana-derived composition according to claim 1, wherein
the step (3) deactivates the proteolytic enzyme with alcohol having a carbon number of 1 to 4, and includes the steps of:
(i) adding the alcohol having the carbon number of 1 to 4 to the enzymatic reactant, and subsequently subjecting a resulting alcohol-added substance to solid-liquid separation to extract a liquid phase; and
(ii) removing the alcohol having the carbon number of 1 to 4 from the liquid phase.

5. The method of producing a banana-derived composition according to claim 4, wherein
the alcohol having the carbon number of 1 to 4 comprises at least one of methanol and ethanol.

6. The method of producing a banana-derived composition according to claim 4, wherein
the step (ii) comprises the step of removing the alcohol having the carbon number of 1 to 4 from the liquid phase by freeze drying.

7. The method of producing a banana-derived composition according to claim 1, further comprising the step of:
(4) desugaring and desalting the composition A to obtain a composition B.

8. The method of producing a banana-derived composition according to claim 1, wherein
the banana-derived composition exhibits biological activity.

9. A biologically active substance, comprising:
as an active ingredient, a banana-derived composition obtained from a flesh part of a banana.

10. The biologically active substance according to claim 9, wherein
the banana-derived composition is obtained from a flesh part of an unripe banana.

11. The biologically active substance according to claim 9, wherein
the banana-derived composition is obtained by adding a proteolytic enzyme to the flesh part of the banana to decompose proteins contained in the flesh part of the banana through an enzyme reaction, and subsequently deactivating the proteolytic enzyme by heating or treating with alcohol.

12. The biologically active substance according to claim 9, wherein
the banana-derived composition contains an amino acid and a tryptophan derivative, a total amount of the amino acid and the tryptophan derivative being 7% by mass or more.

13. The biologically active substance according to claim 12, wherein
the amino acid includes phenylalanine and tryptophan, and the tryptophan derivative includes serotonin.

14. The biologically active substance according to claim 9, wherein
the biologically active substance exhibits an action of inhibiting a blood sugar level rise.

15. The biologically active substance according to claim 9, wherein
the biologically active substance exhibits an action of promoting collagen production.
